(19) United States
(12) Patent Application Publication
(10) Pub. No.: US 2022/0180509 A1
(43) Pub. Date: Jun. 9, 2022

(54) DIAGNOSTIC TOOL FOR EYE DISEASE DETECTION

(71) Applicant: TEXAS TECH UNIVERSITY SYSTEM, Lubbock, TX (US)

(72) Inventors: Jo Woon Chong, Lubbock, TX (US); Behnam Askarian, Lubbock, TX (US); Fatemehsadat Tabei, Lubbock, TX (US)

(73) Assignee: TEXAS TECH UNIVERSITY SYSTEM, Lubbock, TX (US)

(21) Appl. No.: 17/437,022

(22) PCT Filed: Mar. 6, 2020
(86) PCT No.: PCT/US20/21574

§ 371 Date: Sep. 7, 2021

Related U.S. Application Data
(60) Provisional application No. 62/814,851, filed on Mar. 6, 2019.

(51) Int. Cl. G06T 7/00; A61B 3/107; A61B 3/14; G06T 7/13; G06T 7/64

(52) U.S. Cl. CPC G06T 7/0012; A61B 3/107; A61B 3/14; G06T 2207/10024; G06T 7/64; G06T 2207/30041; G06T 7/13

(57) ABSTRACT

Diagnostic system and method for eye disease detection using a smartphone. At least some of the example embodiments are methods including capturing, by way of a camera lens on a device, an image of an eye to create a raw specimen, wherein the image of the eye comprises a series of concentric rings on a cornea of the eye; processing the raw specimen to create a processed specimen; performing edge detection on the processed specimen to detect a boundary of a cornea; determining a topography of the eye based on the series of concentric rings; and classifying the processed specimen as including an eye disease, based on the determining the topography.

### CROSS-REFERENCE TO RELATED APPLICATIONS

This application claims priority to U.S. Patent Application Ser. No. 62/814,851, filed on Mar. 6, 2019, entitled “DIAGNOSTIC TOOL FOR EYE DISEASE DETECTION”, which is hereby incorporated herein by reference in its entirety for all purposes.

### BACKGROUND

All too often, as we age, our eyesight deteriorates. According to the Centers for Disease Control and Prevention (CDC), in a population of Americans over 40, 16% have cataracts and 2% have glaucoma. A more rare disease is keratoconus, which is an irregularity of the shape of the cornea. Keratoconus is treatable in early stages, however, keratoconus is often detected using bulky and expensive machines operated by trained technicians. For example, eye diseases can be detected by OCT, UBM, corneal topography, Scheimpflug camera, laser interferometry, and computerized videokeratoscopy. As topography devices are large, expensive, and not portable, keratoconus may not be detected in early stages, and therefore go untreated, in a poor population or a population that lacks access to healthcare.

### BRIEF DESCRIPTION OF THE DRAWINGS

For a detailed description of example embodiments, reference will now be made to the accompanying drawings in which:

FIG. 1 shows a method, in accordance with at least some embodiments.

FIGS. 2A, 2B, and 2C shows an example apparatus, in accordance with at least some embodiment.

FIG. 3 shows an example apparatus, in accordance with at least some embodiments.

FIGS. 4A and 4B show a front elevation view of the apparatus, in accordance with at least some embodiments.

FIG. 5 shows a side elevation view of an apparatus coupled to a device, in accordance with at least some embodiments.

FIGS. 6A and 6B show a screen capture of a device, in accordance with at least some embodiments.

FIGS. 7A and 7B show a screen capture of a device and a front elevation view of the apparatus, in accordance with at least some embodiments.

FIGS. 8A and 8B show a screen capture of a device, in accordance with at least some embodiments.

FIGS. 9A and 9B show a screen capture of a device, in accordance with at least some embodiments.

FIG. 10A shows a front elevation view of an apparatus.

FIG. 10B shows a side elevation view of a manner in which light from an apparatus is processed, in accordance with at least some embodiments.

FIG. 11 shows a mathematical relationship, in accordance with at least some embodiments.

FIGS. 12A and 12B show a screen capture of a device, in accordance with at least some embodiments.

FIGS. 13A and 13B show a screen capture of a device, in accordance with at least some embodiments.

FIGS. 14A and 14B show topographical maps, in accordance with at least some embodiments.

FIGS. 15A and 15B show an output of a program, in accordance with at least some embodiments.

FIG. 16 illustrates an example computer system.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Before describing the embodiments of the present invention, definitions are set forth of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

“Controller” shall mean individual circuit components on a substrate, an application specific integrated circuit (ASIC) constructed on a substrate, a microcontroller constructed on a substrate (with controlling software stored on the substrate), or combinations thereof, configured to read signals and take action responsive to such signals.

The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code.

Definitions for other certain words and phrases are provided throughout this patent document. Those of ordinary skill in the art should understand that in many if not most instances, such definitions apply to prior as well as future uses of such defined words and phrases.

Often, eye diseases such as keratoconus is detected in clinics with ophthalmic devices, which are large, expensive, not portable, and are operated by trained technicians. At least some of the example embodiments are directed to a diagnostic tool for eye disease detection using a smartphone. More particularly, an affordable and easy-to-use diagnostic tool for eye disease detection, such as an eye disease detection application operating on a smart phone. The eye disease detection application is configured to detect diseases such as keratoconus, cataract, glaucoma, and strabismus. The specification now turns to an example current sense system in accordance with example embodiments.

It is therefore an embodiment of the present invention to provide a method A method of screening for eye disease, comprising: capturing, by way of a camera lens on a device, an image of an eye to create a raw specimen, wherein the image of the eye comprises a series of concentric rings on a cornea of the eye; processing the raw specimen to create a processed specimen; performing edge detection on the processed specimen to detect a boundary of a cornea; determining a topography of the eye based on the series of concentric rings presented in the processed specimen; and classifying the processed specimen as including an eye disease, based on the topography of the eye.

In one embodiment the raw specimen obtained is processed by the device, further comprising: cropping unnecessary areas in the image; filtering image noise; and converting the image from color (RGB) to grayscale. In another embodiment, determining the topography of the eye further comprises: executing a Canny algorithm to detect the edges of the cornea; executing an algorithm to detect the reflected Placido's disks; and computing the curvature of the cornea and generating a topographic map of the cornea. In another embodiment coupling an apparatus to the device, such that a distal end of the apparatus telescopes over the camera lens. In another coupling the apparatus to the device further comprises coupling the distal end of the apparatus by way of a clip configured to clamp a top portion of the device, wherein the clip is coupled to the distal end of the apparatus.

In another embodiment capturing by way of the camera lens on the device further comprises: placing a proximal end of the apparatus over the eye; and transmitting a light through the apparatus onto the eye, wherein the apparatus comprises Placido's disks configured to project the series of concentric rings on the cornea of the eye. In one aspect the topography comprises a sagittal curvature map. In another aspect the processed image presents a k-value and elevational map. K-value is calculated using the following formula (Formula I):

$$K ( dpt ) = \frac{{n}_{2} - {n}_{1}}{{R}_{a n terior}} \times 1000 \qquad Formula I$$

wherein n₁=refractive index of air; and

wherein n₂=refractive index of the corneal power.

In another embodiment, an apparatus is provided that is configured to couple to a device and used to diagnose an eye disease, the apparatus comprising: a distal end configured to couple to a camera lens of the device; a proximal end configured to telescope around an eye; and a cone-shaped middle portion comprising Placido's disks, wherein the Placido's disks are configured to project a series of concentric rings on a cornea of an eye, and wherein a lip of the middle portion forms the proximal end of the apparatus. In one aspect the device is a smartphone executing a mobile application programmed for determining an eye disease based on the topography of the eye. In another embodiment the apparatus comprises a second middle portion shaped as a cylindrical tube, where one end of the cylindrical tube forms the distal end of the apparatus. In another embodiment the apparatus comprises a clamping device coupled to the distal end and configured to clamp the apparatus to the smartphone. In one embodiment a power source coupled to the apparatus, which may be from the device itself, via a smartphone tethered to the apparatus. In another embodiment the apparatus has its own power source, including a battery.

It is another embodiment of the present invention to provide a system for diagnosis of an eye disease comprising: a device comprising a processor and a camera; an apparatus comprising a distal end configured to couple to a camera lens of the device comprising a proximal end configured to telescope around an eye and a cone-shaped middle portion comprising Placido's disks, wherein the Placido's disks are configured to project a series of concentric rings on a cornea of an eye, and wherein a lip of the middle portion forms the proximal end of the apparatus; and a memory configured to store instructions that, when executed by the processor on the device, cause the processor to: capture, by way of the camera lens on a device, an image of an eye to create a raw specimen, wherein the image of the eye comprises a series of concentric rings on a cornea of the eye; process the raw specimen to create a processed specimen; perform edge detection on the processed specimen to detect a boundary of a cornea; determine a topography of the eye based on the series of concentric rings presented in the processed specimen; and classify the processed specimen as including or not including an eye disease, based on the topography of the eye.

In one embodiment the device comprising the processor and the camera comprises a smartphone executing a mobile application programmed for determining an eye disease based on the topography of the eye. In another embodiment the apparatus further comprises a second middle portion shaped as a cylindrical tube, where one end of the cylindrical tube forms the distal end of the apparatus.

In another embodiment, processing the raw specimen further comprises: cropping unnecessary areas in the image; filtering image noise; and converting the image from color (RGB) to grayscale. In another embodiment, determining the topography of the eye further comprises: executing a Canny algorithm to detect the edges of the cornea; executing an algorithm to detect the reflected Placido's disks; and computing the curvature of the cornea and generating a topographic map of the cornea.

In one embodiment, the present invention is capable of coupling the apparatus to the device, such that a distal end of the apparatus telescopes over the camera lens of the device. Coupling the apparatus to the device may further comprise coupling the distal end of the apparatus by way of a clip configured to clamp a top portion of the device, wherein the clip is coupled to the distal end of the apparatus.

In another embodiment the system is capable of capturing by way of the camera lens on the device which further comprises: placing a proximal end of the apparatus over the eye; and transmitting a light through the apparatus onto the eye, wherein the apparatus comprises Placido's disks configured to project the series of concentric rings on the cornea of the eye. In one embodiment, the topography comprises a sagittal curvature map. In another embodiment, the processed image presents a k-value and elevational map.

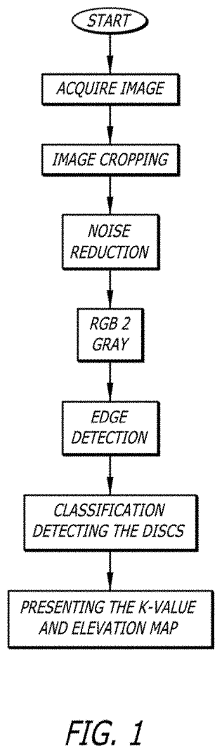

FIGS. 1 through 16, discussed below, and the various embodiments used to describe the principles of this disclosure are by way of illustration only and should not be construed in any way to limit the scope of the disclosure

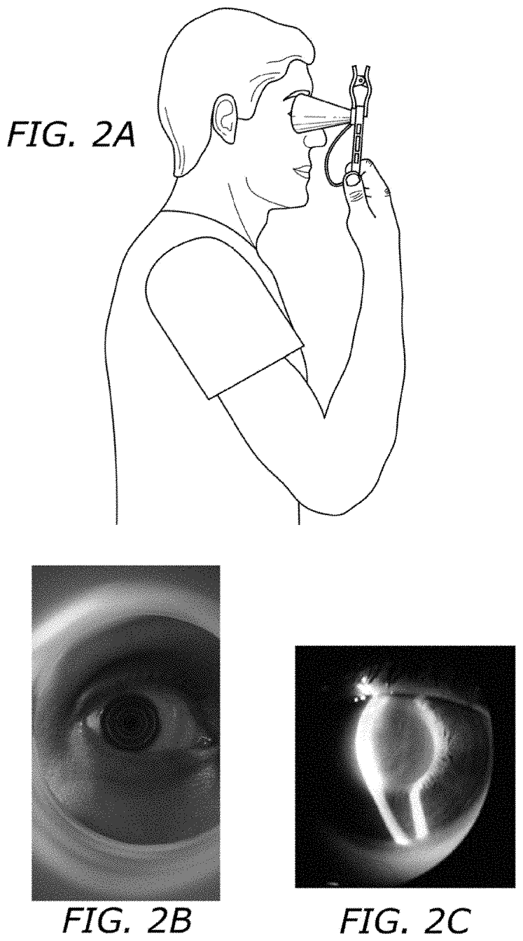

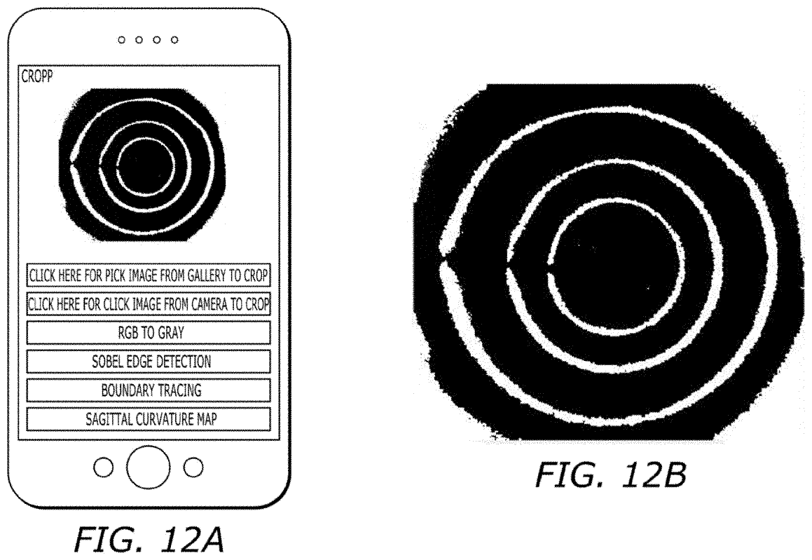

FIG. 1 shows a method, in accordance with at least some embodiments, including being carried out by the system including an apparatus and device set forth in FIGS. 2A through 10B, as well as FIGS. 12A through 16. The human eye includes the cornea (transparent layer), the crystalline lens, and the iris. The cornea is the transparent portion of the eye that covers the iris, pupil, and the inner fluid-filled space inside the eye. The cornea is largely responsible for focusing (i.e., refracting) the light entering the eye (changing the direction the light travels), accounting for approximately two-thirds of the eye's ability to refract light (refractive powers). When an eye has too much too little refractive power refractive error occurs—resulting in vision problems (e.g., near-sighted or far-sighted). The cornea is susceptible to developing disorders that may severely impair its function, such as losing transparency, losing its shape, or loss of oxygen supply.

Medical professional utilize corneal topography systems to analyze the structure of the eye. The general function of these devices is to project a light pattern on the surface of the patient's cornea and capture the reflection on a camera. The pattern that is reflected back to the camera is the shape of the patient's cornea, analogous to a topographic map representing the various dimensions of an area. Currently, most corneal topography systems are constructed with sophisticated, expensive components that result in costly prices.

The disclosed apparatus and method herein provides sufficient diagnostics while utilizing cheaper and portable components. Accordingly a method and device are disclosed for analyzing a patient's cornea by attaching a hardware lens to a smartphone. The smartphone is positioned in front of the patient's eye and executes a software that projects light onto the cornea and analyzes the reflected image (Placido's disks). The software accomplishes this by: (1) cropping unnecessary areas in the image; (2) filtering image noise; (3) converting the image from color (RGB) to grayscale; (4) executing a Canny algorithm to detect the edges of the cornea; (5) executing an algorithm to detect the reflected Placido's disks; and (6) computing the curvature of the cornea and generating a topographic map of the cornea.

FIG. 1 illustrates an example method, in accordance with some embodiments, used to detect keratoconus. Presently, keratoconus is detected by one of the following laboratory or clinical methods: optical coherence tomography (OCT), ultrasound bio-microscopy (UBM), corneal topography, Scheimpflug camera, and laser interferometry. These methods include projecting light circles (known as Placido's disks) on the surface of the cornea, and measuring the differences between the reference and reflected circles. Accordingly, the corneal topography detects any irregularities in a cornea's shape. The automated instrument can produce color-coded contour maps of the eye's topography or even three-dimensional visualizations of its surface.

Placido's's disks are often presented via a keratoscope, an ophthalmic instrument used to assess the shape of the anterior surface of the cornea. A series of concentric rings is projected onto the cornea and their reflection viewed by the examiner through a small hole in the center of the disk. Placido's's disk was a major advancement in the late 19th century. Placido's disk has stood the test of time and the current Placido's based topographers work on the same principle of assessing the reflection of a concentric set of black and white rings from the convex anterior surface of the cornea (see FIG. 12B).

As is known, symptoms of keratoconus include a thinner middle cornea which bulges outward gradually, causing the cornea to change shape into a cone-shaped cornea (as can be seen in the processed specimen in FIG. 2B). Depending on the thickness, steepness, and morphology of the cornea, keratoconus is classified into four stages: mild, moderate, advanced, and severe stages. If caught in the early stages, keratoconus can be effectively treated by treatments including corneal collagen cross-linking.

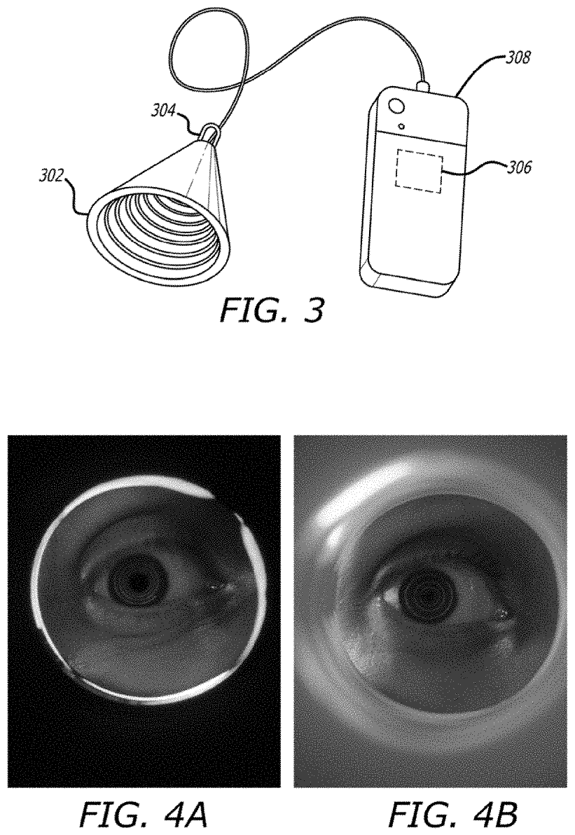

However, such methods use large and expensive equipment. Accordingly, cost and access to affordable healthcare can be a barrier to early detection of keratoconus. The described diagnostic application 118 provides a low-cost method for detecting keratoconus that uses a smartphone and apparatus as shown in FIG. 3.

FIGS. 2A, 2B, and 2C shows an example apparatus, its positioning and orientation to a patient's eye, and method for acquiring an image, in accordance with at least some embodiment. Specifically, in FIGS. 2A, 2B, and 2C an image of the eye is acquired (Placido's disk and slit lamp on cornea) as set forth in FIG. 2B (front view) and FIG. 2C (side view).

FIG. 3 shows an example apparatus, in accordance with at least some embodiments. The apparatus has a cone shape, where the wider side of the cone (proximal end 302) that is placed around the eye. In some embodiments, the apparatus, has a radius (of the wider side, proximal end 302) between 1-2 inches, and a length (measured from a proximal end of the apparatus to a distal end 304 between 1-6 inches. In other embodiments, the proximal end 302 of the apparatus has a radius between 0.2 to 1.5 inches and a length measured from a proximal end 302 of the apparatus to a distal end 304 between 2-10 inches.

Additionally, the Placido's disk is coupled to a power source such as a battery compartment. The cone may be communicatively coupled to a computing device 308 (e.g., smartphone). The computing device 308 may include a processing device 306, a memory device, and/or a network device. The memory device may store instructions that implement any of the methodologies, functions, or operations described herein. The processing device may be communicatively coupled to the memory device and may execute the instructions to perform any of the methodologies, functions, or operations described herein. The term “controller” and “processing device” may be used interchangeably herein.

FIGS. 4A and 4B show a front elevation view of the apparatus, in accordance with at least some embodiments. In particular, the elevation view shows the series of concentric rings (the Placido's disks) projected onto a cornea, where the reflection can be captured by a smartphone (e.g., by way of a camera lens coupled to the processing device 306 of the smartphone) and assessed.

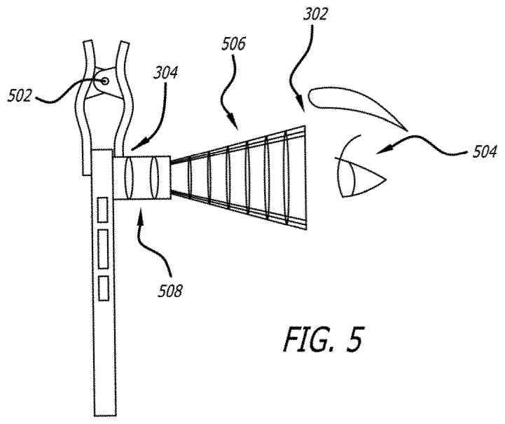

FIG. 5 shows a side elevation view of an apparatus coupled to a device, in accordance with at least some embodiments. In the present embodiment, the device is a smartphone. The apparatus includes an adjustable LED light acting as a slit lamp coupled to a Placido's disk within the cone 506, capable of illumination in order to project the Placido's disk rings onto the cornea. The cone 506 apparatus is coupled at the distal end 304 to a smartphone, over the cameral of the smartphone, by a clamping device 502. The proximal end 302 is thereafter capable of being presented to the patient's eye 504. The patient may self-administer the system

The apparatus is configured to couple to a smartphone and used to diagnose an eye disease. The apparatus includes a distal end 304 configured to couple to a camera lens of the smartphone. A proximal end 302 of the apparatus is configured to telescope around the eye 504. The apparatus also includes a cone-shaped middle portion 506 that includes Placido's disks, where the Placido's disks are configured to project a series of concentric rings on a cornea of the eye 504. The lip of the middle portion forms the proximal end of the apparatus.

The middle portion 506 also couples a second middle portion 508 shaped as a cylindrical tube, where one end of the cylindrical tube forms the distal end 304 of the apparatus.

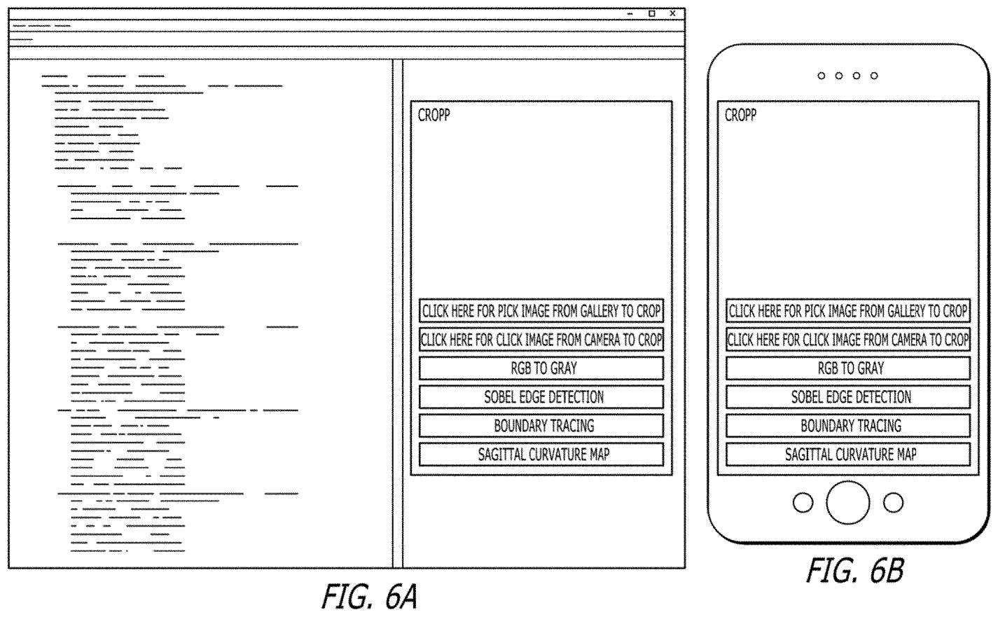

FIGS. 6A and 6B show a screen capture of a device, in accordance with at least some embodiments. In various embodiments, the smart phone is configured to execute a diagnostic application (e.g., Android app, iOS app). The diagnostic application, is capable of providing multiple elective steps including (1) cropping from a gallery; (2) cropping from an image captured from a camera; (3) RGB to grayscale; (4) Soble edge detection; (5) boundary tracing; and (6) preparation of a sagittal curvature map.

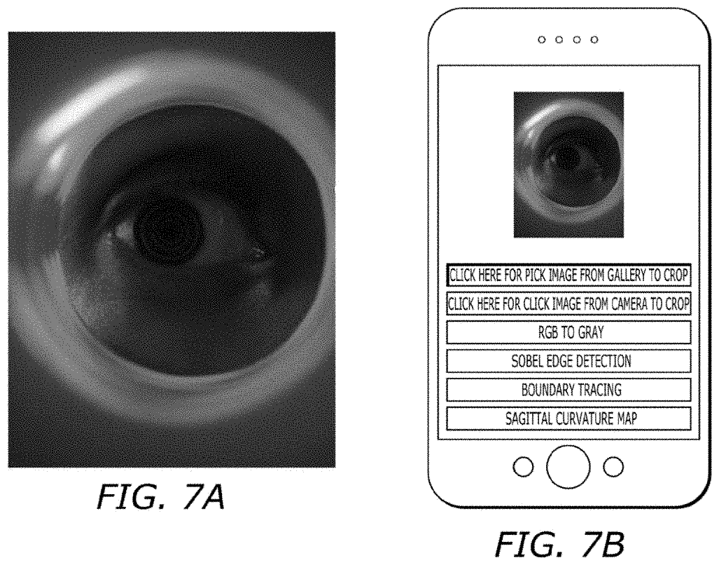

FIG. 7A shows a screen capture of a device and a front elevation view of the apparatus, in accordance with at least some embodiments. In FIG. 7B, the diagnostic application crops the captured image of FIG. 7A, which is then capable of being processed by the device

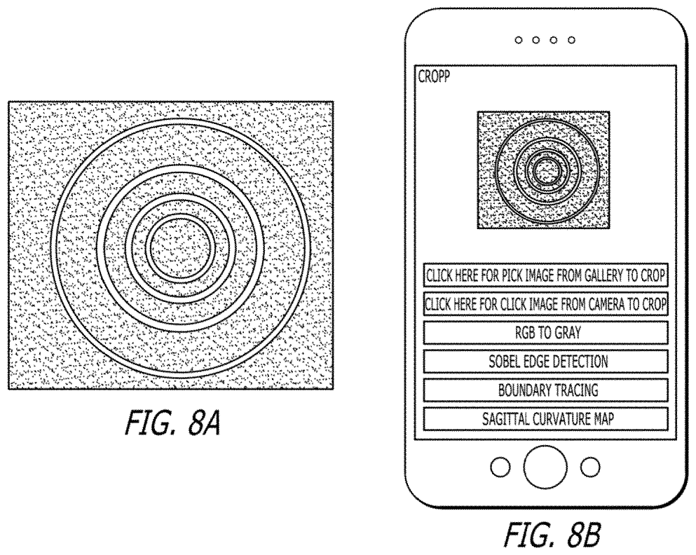

FIG. 8A shows a screen capture of a device, in accordance with at least some embodiments. The screen capture has been processed to identify the Placido's disk obtained by sobel edge detection capability, thus emphasizing the captured rings presented on the image. The image is further capable of being processed to determine topography via the device pursuant to FIG. 8B.

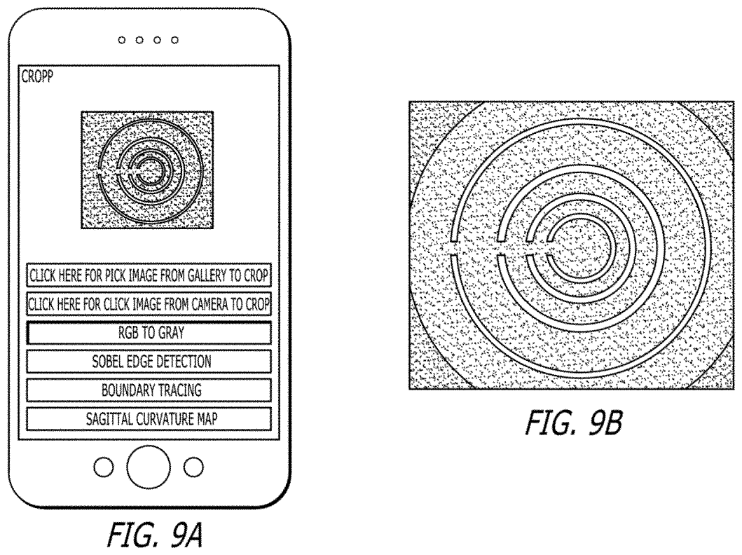

FIG. 9B shows a screen capture of a device, in accordance with at least some embodiments. In FIG. 9A, the image of FIG. 9B is converted from a RGB format to grayscale.

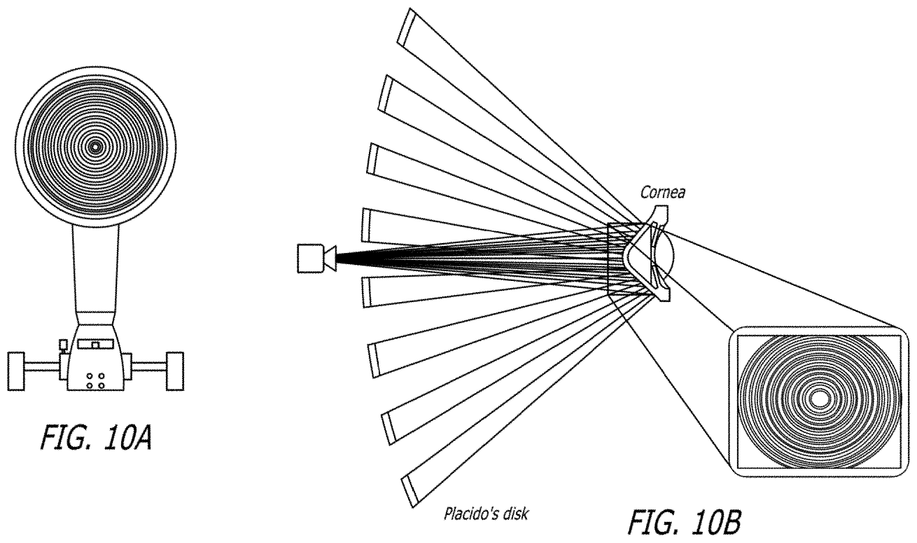

FIG. 10A shows a front elevation view of an apparatus having the proximal end presented showing the Placido's disk features of the apparatus. The accompanying clamp for affixing the apparatus is shown, which, when positioned, allows for the apparatus, via the distal end, to be positioned over the camera of a device.

FIG. 10B shows a side elevation view of a manner in which light from an apparatus is processed, in accordance with at least some embodiments. Utilizing the Placido's disk apparatus, various rings of differing diameters around the cornea are illuminated and captured via a camera on a device. The rings are then collected as shown in the exploded image of FIG. 10B. The image is thereafter processed according to some embodiments for creating a topography map capable of being used for diagnosing certain eye disease.

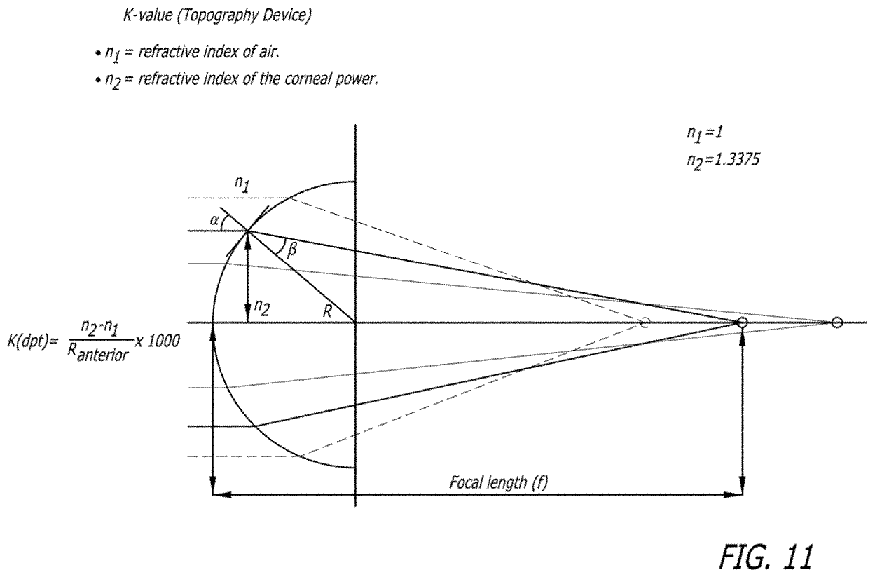

FIG. 11 shows a mathematical relationship, in accordance with at least some embodiments, wherein the processed image presents a k-value and elevational map. K-value is calculated using the Formula I (previously presented).

FIG. 12A shows a screen capture of a device, in accordance with at least some embodiments. In particular, the diagnostic application is configured to implement noise reduction and grayscale to binary sobel filter as shown in FIG. 12B.

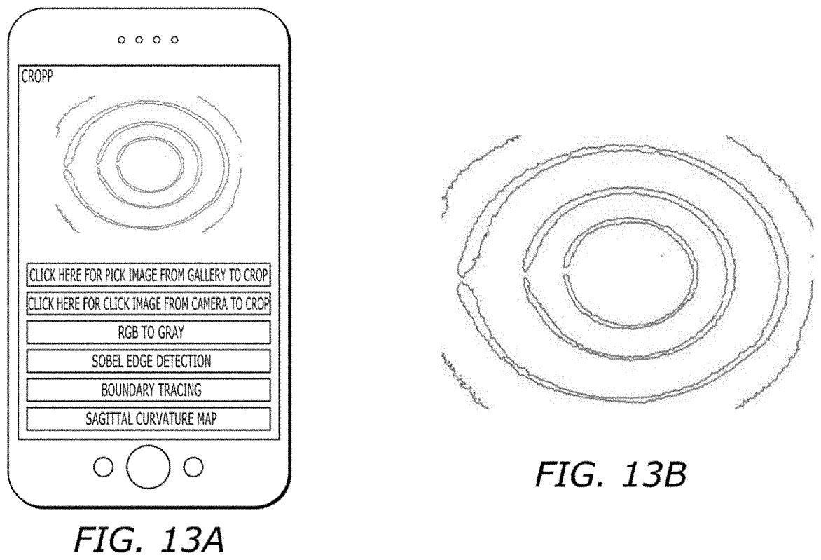

FIG. 13A shows a screen capture of a device, in accordance with at least some embodiments. The diagnostic application performs boundary tracing. In various embodiments, the grayscale images are binary images upon which the diagnostic application performs additional processing that includes edge detection algorithms and morphological dilation, as shown in FIG. 13B.

For example, the diagnostic application can perform morphological dilation to generate a smooth edge of a boundary. Any dilation algorithm can be used (e.g., morphological dilation) that closes gaps/fills holes in discontinuous objects to produce a more connected structure. Morphological dilation is a morphological operation implemented on binary images, such as processed specimens in FIGS. 3C and 3D, where dilations add pixels to the pixels in the identified boundary (e.g., boundaries 302 or 304) to generate a smoother boundary.

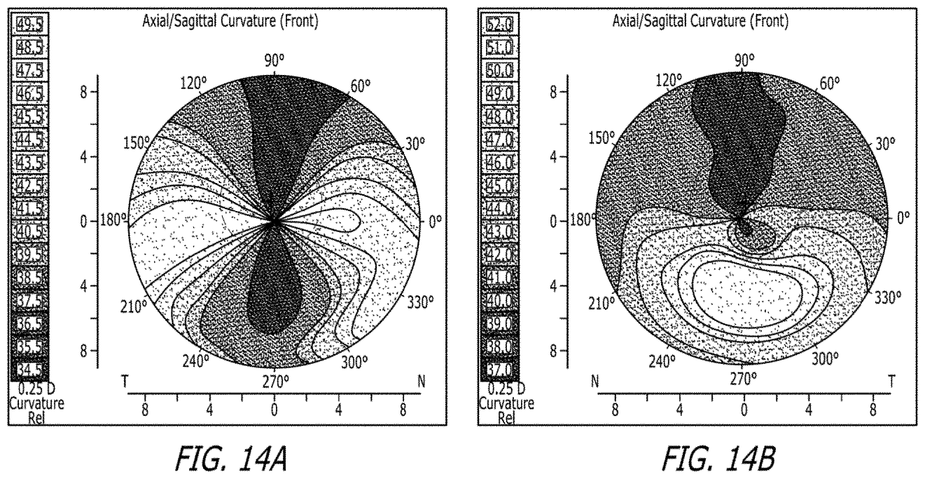

FIGS. 14A and 14B show topographical maps, in accordance with at least some embodiments. In particular, FIGS. 14A and 14B illustrate a Sagittal Curvature Map.

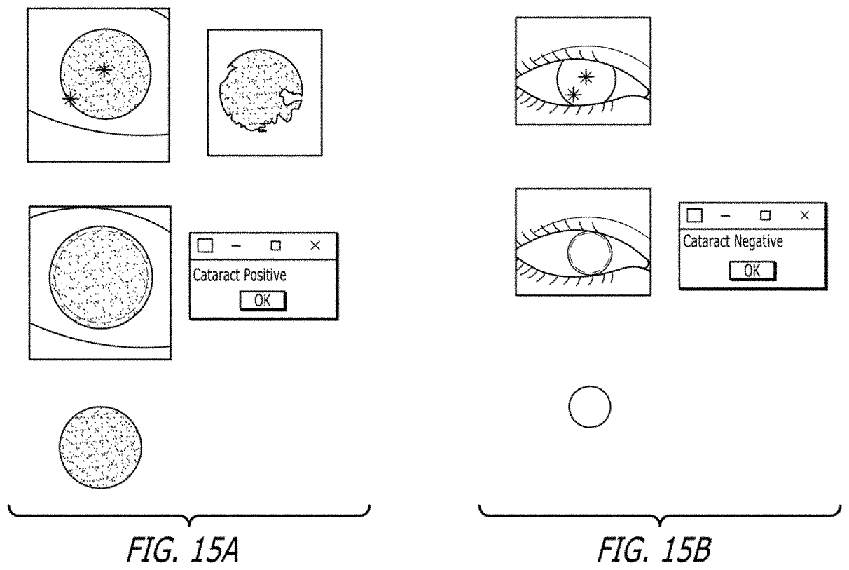

FIGS. 15A and 15B show an output of a program, in accordance with at least some embodiments, for cataract detection. The topography detected using the present invention, and while not always routine using traditional methods, assessment of the corneal contour using topography is useful to determine whether irregularities in corneal power and shape are contributing to visual impairment. The present invention will also be helpful prior to cataract surgery to evaluate foveal architecture or to identify the presence of concomitant retinal disease and anterior segment disorders, such as posterior polar cataracts, even when the foveal center and immediately surrounding areas appear normal on direct examination.

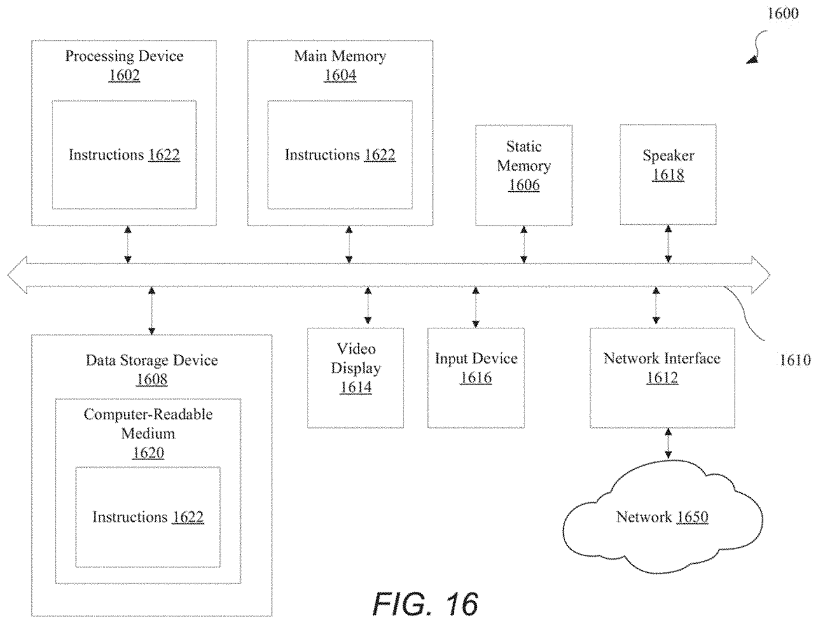

FIG. 16 illustrates an example computer system 1600, which can perform any one or more of the methods described herein. In one example, computer system 1600 may correspond to the computing device 308 of FIG. 3. The computer system 1600 may be connected (e.g., networked) to other computer systems in a LAN, an intranet, an extranet, or the Internet. The computer system 1600 may be a personal computer (PC), a tablet computer, a wearable (e.g., wristband), a set-top box (STB), a personal Digital Assistant (PDA), a mobile phone (smartphone), a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1600 includes a processing device 1602, a main memory 1604 (e.g., read-only memory (ROM), solid state drive (SSD), flash memory, dynamic random access memory (DRAM) such as synchronous DRAM (SDRAM)), a static memory 1606 (e.g., solid state drive (SSD), flash memory, static random access memory (SRAM)), and a data storage device 1608, which communicate with each other via a bus 1610.

Processing device 1602 represents one or more general-purpose processing devices such as a microprocessor, central processing unit, or the like. More particularly, the processing device 1602 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1602 may also be one or more special-purpose processing devices such as an application specific integrated circuit (ASIC), a field programmable gate array (FPGA), a digital signal processor (DSP), network processor, or the like. The processing device 1602 is configured to execute instructions for performing any of the operations and steps discussed herein.

The computer system 1600 may further include a network interface device 1612. The computer system 1600 also may include a video display 1614 (e.g., a liquid crystal display (LCD) or a cathode ray tube (CRT)), one or more input devices 1616 (e.g., a keyboard and/or a mouse), and one or more speakers 1618 (e.g., a speaker). In one illustrative example, the video display 1614 and the input device(s) 1616 may be combined into a single component or device (e.g., an LCD touch screen).

The data storage device 1616 may include a computer-readable medium 1620 on which the instructions 1622 embodying any one or more of the methodologies, functions, or operations described herein are stored. The instructions 1622 may also reside, completely or at least partially, within the main memory 1604 and/or within the processing device 1602 during execution thereof by the computer system 1600. As such, the main memory 1604 and the processing device 1602 also constitute computer-readable media. The instructions 1622 may further be transmitted or received over a network 1650 via the network interface device 1612.

While the computer-readable storage medium 1620 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications. None of the descriptions in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle.

1. A method of determining the presence of eye disease, comprising:
capturing, by way of a camera lens on a device, an image of an eye to create a raw specimen, wherein the image of the eye comprises a series of concentric rings on a cornea of the eye;
processing the raw specimen to create a processed specimen that presents a k-value and elevational map;
performing edge detection on the processed specimen to detect a boundary of a cornea;
determining a topography of the eye comprising a sagittal curvature map based on the series of concentric rings presented in the processed specimen; and
classifying the processed specimen as including an eye disease, based on the topography of the eye.

2. The method of claim 1, wherein the raw specimen is processed by the device, further comprising:
cropping unnecessary areas in the image;
filtering image noise; and
converting the image from color (RGB) to grayscale.

3. The method of claim 1, wherein determining the topography of the eye further comprises:
executing a Canny algorithm to detect the edges of the cornea;
executing an algorithm to detect the reflected Placido's disks; and
computing the curvature of the cornea and generating a topographic map of the cornea.

4. The method of claim 1, further comprising coupling an apparatus to the device, such that a distal end of the apparatus telescopes over the camera lens.

5. The method of claim 4, wherein coupling the apparatus to the device further comprises coupling the distal end of the apparatus by way of a clip configured to clamp a top portion of the device, wherein the clip is coupled to the distal end of the apparatus.

6. The method of claim 4, wherein capturing by way of the camera lens on the device further comprises:
placing a proximal end of the apparatus over the eye; and
transmitting a light through the apparatus onto the eye, wherein the apparatus comprises Placido's disks configured to project the series of concentric rings on the cornea of the eye.

7. The method of claim 1, wherein the topography comprises a sagittal curvature map.

8. The method of claim 1, wherein the processed image presents a k-value and elevational map.

9. An apparatus configured to couple to a device and used to determine the presence of an eye disease, the apparatus comprising:
a distal end configured to couple to a camera lens of the device;
a proximal end configured to telescope around an eye; and
a cone-shaped middle portion comprising Placido's disks,
i. wherein the Placido's disks are configured to project a series of concentric rings on a cornea of an eye capable of generating a processed specimen that presents a k-value and elevational map,
ii. wherein a lip of the middle portion forms the proximal end of the apparatus.

10. The apparatus of claim 9, wherein the device is a smartphone executing a mobile application programmed for determining an eye disease based on the topography of the eye comprising a sagittal curvature map.

11. The apparatus of claim 9, further comprising a second middle portion shaped as a cylindrical tube, where one end of the cylindrical tube forms the distal end of the apparatus.

12. The apparatus of claim 10, further comprising a clamping device coupled to the distal end and configured to clamp the apparatus to the smartphone.

13. The apparatus of claim 9, wherein the proximal end width is between 1 and 3 inches.

14. The apparatus of claim 9, wherein the length of the apparatus from the proximal end to the distal end is between 1 and 10 inches.

15. The apparatus of claim 9, wherein the radius of the proximal end is between 0.2 and 1.5 inches.

16. The apparatus of claim 9, further comprising a power source coupled to the apparatus.

17. The apparatus of claim 16, wherein the power source coupled to the Placido's disks is the device.

18. A system for diagnosis of an eye disease comprising:
a device comprising a processor and a camera;
an apparatus comprising a distal end configured to couple to a camera lens of the device comprising a proximal end configured to telescope around an eye and a cone-shaped middle portion comprising Placido's disks, wherein the Placido's disks are configured to project a series of concentric rings on a cornea of an eye, and wherein a lip of the middle portion forms the proximal end of the apparatus; and
a memory configured to store instructions that, when executed by the processor on the device, cause the processor to: capture, by way of the camera lens on a device, an image of an eye to create a raw specimen, wherein the image of the eye comprises a series of concentric rings on a cornea of the eye; process the raw specimen to create a processed specimen that presents a k-value and elevational map; perform edge detection on the processed specimen to detect a boundary of a cornea; determine a topography of the eye based on the series of concentric rings presented in the processed specimen wherein the topography comprises a sagittal curvature map;
and classify the processed specimen as including or not including an eye disease, based on the topography of the eye.

19. The system of claim 18, wherein the device comprising the processor and the camera comprises a smartphone executing a mobile application programmed for determining an eye disease based on the topography of the eye.

20. The system of claim 18, wherein the apparatus further comprises a second middle portion shaped as a cylindrical tube, where one end of the cylindrical tube forms the distal end of the apparatus.

21. The system of claim 18, wherein processing the raw specimen further comprises:
cropping unnecessary areas in the image;
filtering image noise; and
converting the image from color (RGB) to grayscale.

22. The system of claim 18, wherein determining the topography of the eye further comprises:
executing a Canny algorithm to detect the edges of the cornea;
executing an algorithm to detect the reflected Placido's disks; and
computing the curvature of the cornea and generating a topographic map of the cornea.

23. The system of claim 18, further comprising coupling the apparatus to the device, such that a distal end of the apparatus telescopes over the camera lens of the device.

24. The system of claim 18, wherein coupling the apparatus to the device further comprises coupling the distal end of the apparatus by way of a clip configured to clamp a top portion of the device, wherein the clip is coupled to the distal end of the apparatus.

25. The system of claim 18, wherein capturing by way of the camera lens on the device further comprises:
placing a proximal end of the apparatus over the eye; and
transmitting a light through the apparatus onto the eye, wherein the apparatus comprises Placido's disks configured to project the series of concentric rings on the cornea of the eye.

26. (canceled)

27. (canceled)
